(19) United States
(12) Patent Application Publication
(10) Pub. No.: US 2015/0066116 A1
(43) Pub. Date: Mar. 5, 2015

(54) Blanket

(71) Applicant: IBENA Textilwerke GmbH, Bocholt (DE)

(72) Inventor: Britta Smeulders, Lichtenvoorde (NL)

(21) Appl. No.: 14/455,489

(22) Filed: Aug. 8, 2014

(30) Foreign Application Priority Data
Aug. 9, 2013 (DE) 10 2013 215 780.0

(51) Int. Cl. A61N 5/06; A61D 99/00

(52) U.S. Cl. CPC A61N 5/0625; A61D 99/00; A61N 2005/063; A61N 2005/0643
USPC 607/90

(57) ABSTRACT

A wellness and/or heat blanket has autonomous, optically conductive fibers which are present in a proportion of 40 g/m² or less, preferably 37 g/m² or less and in particular approximately 35 g/m² and/or which constitute a weight proportion of less than 15%, preferably less than 10% and in particular 8% or less.

### TECHNICAL FIELD

The invention relates to a wellness and/or heat blanket which will hereinbelow be designated merely as blanket for the sake of simplicity and which is for general well-being, it having been found for the blanket according to the invention that it serves bioenergetic stimulation and can generate a healing effect.

### PRIOR ART

Blankets are known both for humans as well as for animals, such as e.g. horses, to keep the body of the wearer warm. Moreover, highly varied configurations of healing blankets are known.

For instance, a blanket is apparent from CN 202314978 U, which has glass fibers as light conductors, into which blue light can be fed, to have a healing effect. KR 20030069497 A relates to a similar subject matter.

Moreover, blankets are known that have a high glass fiber proportion e.g. to guarantee a particular refractoriness.

### DESCRIPTION OF THE INVENTION

The invention is based on the object to create a wellness and/or heat blanket having an improved effect.

This object is solved by the blanket described in claim 1.

Accordingly, this blanket has autonomous, optically conductive fibers with a weight proportion of less than 15%, preferably less than 10% and in particular 8% or less, and particularly preferably approximately 7%. Alternatively or additionally, the proportion of the optically conductive fibers is 40 g/m² or less, preferably 37 g/m² or less and in particular approximately 35 g/m². “Autonomous”, optically conductive fibers are to be understood such that these do not have a connector or the like for feeding in light and/or electric power. On the contrary, according to the invention, as will be described in more detail below, the optically conductive fibers are provided in the yarn used for the blanket and it has been found that a healing effect can thus be achieved. Glass is the preferred material for the optically conductive fibers. However, these can also be made of quartz (SiO2), other materials, acrylate of which e.g. plastic lenses for spectacles are made, or a combination of said materials. For the sake of simplicity, the optically conductive fibers are designated hereinbelow as glass fibers.

Preferred embodiments of the blanket according to the invention are described in the further claims.

In first tests, particularly good experiences were made with glass fibers present as filaments.

For the use of glass fibers, it is furthermore preferred that these are used as core in a so-called core yarn which, in other words, has conventional fibers used for blankets, e.g. of cotton, on its exterior. This results in the advantage that the glass fibers are not visible for the user, rather the optics and haptics of a “conventional” blanket are created. In particular, the blanket according to the invention is thus to the furthest extent a “normal” textile fabric, which is in particular also washable. The brittle, optically conductive fibers that can break easily in particular when glass fibers are used and can therefore lead to itching are advantageously “hidden” inside the blanket. Alternatively or additionally, the optically conductive fibers can be provided in a cover yarn. As a further alternative or addition, the optically conductive fibers can be siliconized or coated in another suitable manner to obtain the impression of a conventional and in particular washable wellness blanket.

In particular, it is presently preferred that the yarn comprising the glass fibers is used as warp yarn. Owing to the fact that the weft yarns can be configured e.g. with face weft and back weft, the warp yarn comprising the optically conductive fibers is covered and held in the interior of the blanket.

It is furthermore advantageous for the healing effect of the blanket according to the invention if the glass fibers have photons, in particular biophotons, stored therein.

The effect of the blanket according to the invention is furthermore particularly extensive if the glass fibers are suited to release photons, in particular biophotons, over a longer period of time. Thus, light although in a hardly visible scope, i.e. light having a wavelength in the range from 200 to 800 nanometers, is emitted.

While no particular requirements are placed on the shape of a blanket suited for humans, it is preferred in an embodiment that the blanket is configured as horse, dog or cat blanket and is accordingly designed and/or dimensioned. Moreover, the blanket according to the invention can be adjusted for any further animals, in particular pets.

1. A wellness and/or heat blanket with autonomous, optically conductive fibers which are present in a proportion of 40 g/m² or less and/or which constitute a weight proportion of less than 15%.

2. The blanket according to claim 1, wherein the optically conductive fibers are present as filaments.

3. The blanket according to claim 1, wherein the optically conductive fibers are provided as core of a core yarn.

4. The blanket according to claim 1, wherein the optically conductive fibers are provided in a warp yarn.

5. The blanket according to claim 1, wherein photon are stored in the optically conductive fibers.

6. The blanket according to claim 1, wherein the optically conductive fibers are configured to release photons.

7. The blanket according to claim 1, wherein it is configured as horse, dog or cat blanket.

8. The blanket according to claim 1, wherein the autonomous, optically conductive fibers are present in a proportion of 37 g/m² or less.

9. The blanket according to claim 1, wherein the autonomous, optically conductive fibers are present in a proportion of approximately 35 g/m².

10. The blanket according to claim 1, wherein the autonomous, optically conductive fibers constitute a weight proportion of less than 10%.

11. The blanket according to claim 1, wherein the autonomous, optically conductive fibers constitute a weight proportion of 8% or less.

12. The blanket according to claim 5, wherein the photons are biophotons.

13. The blanket according to claim 6, wherein the optically conductive fibers are configured to release biophotons.
